(19) United States
(12) Patent Application Publication
(10) Pub. No.: US 2017/0325036 A1
(43) Pub. Date: Nov. 9, 2017

(54) HEARING DEVICE

(71) Applicant: Sonova AG, Stafa (CH)

(72) Inventors: Jan Angst, Hirzel (CH); Erdal Karamuk, Mannedorf (CH); Josef Mueller, Feusisberg (CH)

(21) Appl. No.: 15/517,244

(22) PCT Filed: Oct. 9, 2014
(86) PCT No.: PCT/EP2014/071628

§ 371 Date: Apr. 6, 2017

(51) Int. Cl. H04R 25/00; A61B 5/12

(52) U.S. Cl. CPC H04R 25/70; H04R 25/305; A61B 5/12; H04R 25/505; H04R 25/50

(57) ABSTRACT

A hearing device including a device body to be worn behind the ear of a user. The hearing device includes an amplifier module and a receiver module, an ear canal plug adapted to be worn into the ear canal of the user, and a receiver including an electro-acoustical converter having an acoustical output. The amplifier module and receiver module are detachably attached to each other along the longitudinal extension of the device body.

### TECHNICAL FIELD

The present invention is related to a hearing device.

### BACKGROUND OF THE INVENTION

Hearing devices are typically small ear-level devices used to improve the hearing capability of hearing impaired people. This is achieved by picking up the surrounding sound with a microphone of a hearing device, processing the microphone signal thereby taking into account the hearing impairment of the user of the hearing device and providing the processed sound signal into an ear canal of the user via a miniature loudspeaker, commonly referred to as a receiver.

With the introduction of external receiver products, Receiver-In-Canal (RIC), the portfolio of different electronic modules has increased significantly. On the other hand, RIC hearing devices benefit from the fact of having a receiver positioned far away from the amplifier. Thereby, mechanical vibrations are reduced and acoustic stability is improved. While an RIC solution offers different power levels with the same amplifier, a standard Behind-The-Ear (BTE) hearing device with a built-in receiver does not offer this option. The only possible variation can be achieved by adapting the acoustic sound tube or transmission path to the ear. Known solutions are undamped and damped ear hooks, slim tubes or power tubes.

In conventional Behind-The-Ear (BTE) applications with built-in receivers, for every configuration, for example in view of the power level, battery size, accessories and alternative inputs, one single amplifier or module is needed. Hence, there does not exist a possibility to either increase battery life in using a bigger battery nor in increasing functionality.

It is therefore an object of the present invention to provide a hearing device in which problems in the state of the art are solved.

### SUMMARY OF THE INVENTION

The present invention is directed to a hearing device comprising a device body to be worn behind the ear of a user, comprising an amplifier module and a receiver module, an ear canal plug adapted to be worn into the ear canal of the user, and a receiver comprising an electro-acoustical converter having an acoustical output, wherein said amplifier module and receiver module are detachably attached to each other along the longitudinal extension of the device body.

The hearing device according to the present invention is adapted to be worn behind the ear and comprises at least two modules, which modules, if combined, build the final appearance of the product. The modules are at least composed of the amplifier module and the receiver module detachably attached to each other along the longitudinal extension of the device body. Hence, provided is a hearing device allowing for a modular product architecture. Both modules can be built out of different materials and colors. The amplifier module can comprise an amplifier, a battery, preferably a rechargeable battery, a microphone system and user controls. The receiver module can comprise a receiver, electrical connections (interface) and retention means.

In an embodiment of the proposed hearing device, the receiver is incorporated into the receiver module, wherein the acoustical output of the electro-acoustical converter is connected to the ear canal plug via a sound tubing. This embodiment allows for a modular combination in that the electrical inputs of the receiver are connected to the electrical interface for interfacing the amplifier module and the receiver module allowing for easy attachment or detachment thereof. The acoustical output of the electro-acoustical converter comprised by the receiver is easily connected to the ear canal plug via a sound tubing.

In an alternative embodiment of the proposed hearing device, the receiver is incorporated into the ear canal plug, wherein the acoustical output of the electro-acoustical converter is exposed to the ear canal, and the electrical input thereof is connected to the receiver module by means of electrical wires. Both embodiments allow to easily separate the receiver module from the amplifier module, wherein both modules can still be placed behind the ear. An electrical interface allows to connect the receiver (two or more contacts) to the bottom of the hearing instrument, which builds the support of the amplifier module, for example. This allows either to detach a receiver module with a receiver behind the ear (first embodiment) or a known module respectively external receiver assembly with a receiver in the ear canal (second embodiment). These embodiments allow to know the coupling of the Behind-The-Ear hearing device solution and to use thereof in order to provide an improved adaptation or rather customization.

In an embodiment of the proposed hearing device, the amplifier module and the receiver module are attached to each other such that, if the device body is worn by the user behind the ear, the amplifier module is arranged substantially oriented upwards. Hence, a hearing device is provided which shows acoustic stability since the impact of receiver vibration on the acoustic stability of the hearing system is reduced significantly. Further, advantageously, a pumping effect of the sound tube has less impact on the microphone pickup as it is separated from the housing.

In an embodiment of the proposed hearing device the amplifier module comprises at least one of an amplifier, a battery, a microphone system and user controls. The hearing device according to the present invention allows to obtain a variety of possibilities regarding a flexible combination. In particular, the placement of the electrical connection to the amplifier module at the bottom of the device body offers increased flexibility for a variety of hearing device module combinations. Further, usage of the same amplifier for a Behind-The-Ear hearing device application with acoustic transmission or a Receiver-In-The-Canal hearing device application with electrical wires connected to the receiver mounted to the ear canal plug, which is itself placed in the ear canal, is possible. Advantageously, a variety of functionalities such as user controls, increased battery lifetime to the product can be added easily.

In an embodiment of the proposed hearing device the microphone system comprises at least one microphone disposed substantially on the top of the amplifier module. This embodiment allows for improved directional hearing. Further, mechanical connection of the tubing system can be omitted, which would otherwise use space on top of the ear.

In an embodiment of the proposed hearing device, the receiver module has a customized shape. The customized shape may depend on the individual anatomy of a user's ear. It may also be individually adapted to allow the contemporary placement of further devices in the vicinity of the user's ear. Such further devices may for example be glasses, communication headsets or protection means for the ears or the head of the user. A receiver module with a customized shape allows for more comfort in wearing the hearing device.

In further embodiments the proposed hearing device further comprises at least one sub-microphone mounted to the sound tubing such to receive sound from inside the ear canal and/or from the vicinity of the concha. This placement allows for improved directional hearing.

In an embodiment of the proposed hearing device the receiver module further comprises retention means adapted to detachably attach the receiver module to the amplifier module. The hearing device according to the present invention advantageously provides for modular architecture configuration. Both modules, i.e. the amplifier module and the receiver module, can be easily fixed together or separated from each other by means of the retention means.

In an embodiment of the proposed hearing device the receiver module further comprises electrical connection means interfacing the receiver module with the amplifier module. This embodiment provides a modular product architecture, in which a commonly known electrical connector is replaced by a receiver module, which can solely include electrical connection means interfacing with the amplifier module. As an option, the receiver module can also comprise the receiver including the sound outlet connected to the ear canal plug by means of the sound tubing.

In further embodiments the proposed hearing device further comprises a functionality module detachably attached to at least one of the receiver module and the amplifier module, wherein the functionality module includes a rechargeable system.

It is expressly pointed out that any combination of the above-mentioned embodiments is subject of further possible embodiments. Only those embodiments are excluded that would result in a contradiction.

### BRIEF DESCRIPTION OF THE DRAWINGS

The present invention is further described with reference to the accompanying drawing jointly illustrating various exemplary embodiments, which are to be considered in connection with the following detailed description. What is shown in the figures is:

FIG. 1 is a schematically view of a hearing device according to the present invention in a first embodiment, and

FIG. 2 is a schematically view of a hearing device according to the present invention in a second embodiment.

### DETAILED DESCRIPTION OF THE INVENTION

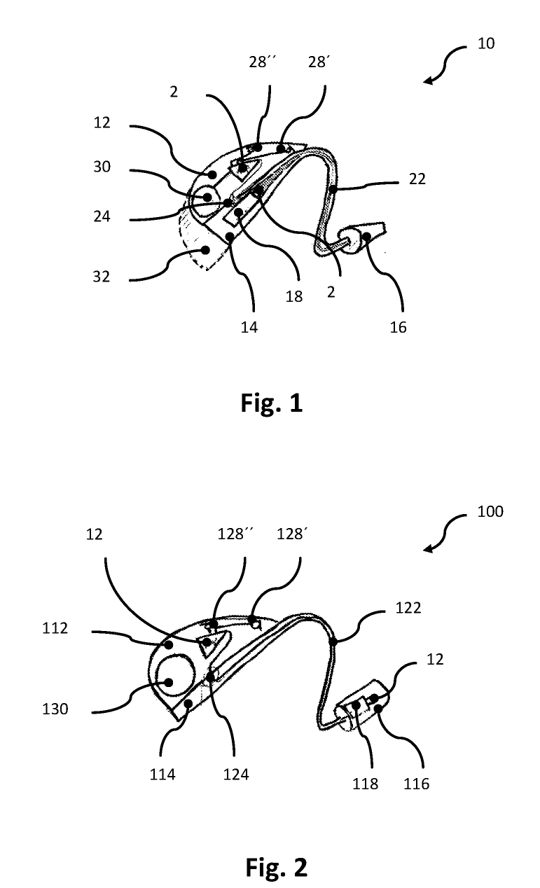

FIG. 1 shows a hearing device 10 according to the present invention in a first embodiment. The hearing device 10 comprises a device body to be worn behind the ear of a user, wherein the device body comprises an amplifier module 12 and a receiver module 14. The hearing device 10 further comprises an ear canal plug 16 adapted to be inserted and worn into the ear canal of the user. The amplifier module 12 and the receiver module 14 are detachably attached to each other along the longitudinal extension of the device body.

The hearing device 10 further comprises a receiver 18 comprising an electro-acoustical converter having an acoustical output 20. In the hearing device according to the first embodiment the receiver 18 is incorporated into the receiver module 14. Further, the acoustical output 20 of the electro-acoustical converter of the receiver 18 is connected to a first end of a sound tubing 22. The other end of the sound tubing 22 is connected to the ear canal plug 16. Advantageously, the receiver 18 is separated from the amplifier module 12, which results in reduced impact of receiver vibration on the acoustic stability of the hearing device 10 as a whole. Further, pumping effect of the sound tubing 22 has reduced impact on the microphone pickup as it is separated from the housing.

The hearing device 10 further comprises an electrical connection means 24 interfacing the receiver module 14 with the amplifier module 12. The electrical connection means 24 allows to detach the receiver module 14 incorporating the receiver 18 from the amplifier module 12.

The amplifier module 12 comprises an amplifier 26, which receives sound signal inputs of microphones 28′, 28″. Said microphones 28′, 28″ are disposed substantially on the top of the amplifier module 12. This localization allows for improved directional hearing since the microphones 28′, 28″ are placed on top of the ear. The signals received from the microphones 28′, 28″ are amplified by the amplifier 26. The amplified electrical signal is output to the electrical connection means 24 connecting the amplifier 26 to the receiver 18. The amplifier module 12 further comprises a battery 30, in particular a rechargeable battery, in order to supply electrical power to the hearing device 10. The hearing device 10 further comprises a functionality module 32 detachably attached to the receiver module 14 and the amplifier module 12. The functionality module 32 can include a rechargeable system, for example. While not shown, the functionality module 32 can be detachably attached to the receiver module 14 or the amplifier module 12.

FIG. 2 shows a hearing device 100 according to the present invention in a second embodiment. The hearing device 100 comprises a device body including an amplifier module 112 and a receiver module 114. The hearing device 100 further comprises an ear canal plug 116 adapted to be worn into the ear canal of the user. Similar to the first embodiment as shown in FIG. 1, said amplifier module 112 and receiver module 114 are detachably attached to each other along the longitudinal extension of the device body.

Further comprised is a receiver 118 including an electro-acoustical converter having an acoustical output 120. According to the second embodiment, said receiver 118 is incorporated into or rather mounted to the ear canal plug 116. The acoustical output 120 of the electro-acoustical converter of the receiver 118 is oriented such to be exposed to the ear canal of the user. The electrical input of the receiver 118 is connected to the receiver module 114 by means of electrical wires 122. One end of the electrical wires 122 is connected to an electrical connection 124 connecting (interfacing) said electrical wires 122 to an amplifier 126. Inputs of said amplifier 126 are connected to respective microphones 128′, 128″ disposed substantially on the top of the amplifier module 112. The amplifier module 112 further comprises a battery 130, in particular a rechargeable battery, in order to supply the hearing device 100 with electrical power.

As shown in FIGS. 1 and 2, both embodiments have in common that the amplifier module 12, 112 and the receiver module 14, 114 are detachably attached to each other along the longitudinal extension of the respective device body. Further, both embodiments have in common that the amplifier module 12, 112 and the receiver module 14, 114 are attached to each other such that, if the device body is worn by the user behind the ear, the amplifier module 12, 112 is arranged substantially oriented upwards. Further, both embodiments have in common that the amplifier module 12, 112 comprises at least one of an amplifier 26, 126, a battery 30, 130, a microphone system 28′, 28″, 128′, 128″ and user controls (not shown). Advantageously, both embodiments have in common that the microphone system 28′, 28″, 128′, 128″, which is composed of a plurality of microphones, is disposed substantially on the top of the respective amplifier module 12, 112.

The present invention provides a fundamentally modular product architecture omitting a commonly known electrical connector for electrical connection of the device body with a receiver fixed to an ear canal plug. According to the present invention, said commonly known electrical connector is replaced by the receiver module 14, 114, which can include only contacts (refer to FIG. 2) but also the whole receiver 18 including the acoustical output 20 (refer to FIG. 1). According to the present invention, each receiver module 14, 114 is optimized for a specific sound delivery system. Hence, commonly known sensors (pressure sensor, push button, etc.) used in the state of the art for recognizing which kind of sound delivery system is mounted onto the device body of the hearing device can be omitted. Therefore, according to the present invention, space required by such sensors can be saved. Further, by omitting such sensors, costs can be reduced as a whole.

According to the present invention, the receiver module 14, 114 can be separated from the amplifier module 12, 112, wherein both modules are still placed behind the ear. This requires an electrical interface for the receiver connection (for example two or more contacts) on the bottom of the hearing instrument which builds the support of the amplifier module 12, 112.

This feature allows either to detach a receiver module with a receiver behind the ear as described relating to the first embodiment and shown in FIG. 1. This feature further allows an external receiver assembly with a receiver in the ear canal as described relating to the second embodiment and shown in FIG. 2.

With the continuous development of rechargeable batteries and respective charging systems for hearing instruments, the need for moving parts such as battery doors is lessen. On the other hand, new wireless technologies in the hearing instrument (for example 2.4 GHz) offer new design rules for relative distances and orientation between single components. According to the present invention, a more modular and flexible product architecture can be realized which allows to optimize critical sub-systems or modules such as the amplifier module to be manufactured in high volumes with a high degree of automation. Hence, manufacturing costs can be reduced.

The present invention further provides increased robustness of the tube interface. The mechanical interface to the sound transmission system (tube or hug, for example) can be solved much more solid as it is not placed on a most sensitive position regarding visibility on the ear.

The inventive module combination offers improved flexibility, which offers a benefit for the fitter at the point of sale (fitter's office). Further, the present invention provides improved acoustic stability and mechanical robustness, which offer increased customer satisfaction.

What is claimed is:

1. A hearing device (10; 100) comprising:
a device body to be worn behind the ear of a user, comprising an amplifier module (12; 112) and a receiver module (14; 114),
an ear canal plug (16; 116) adapted to be worn into the ear canal of the user, and
a receiver (18; 118) comprising an electro-acoustical converter having an acoustical output (20; 120),
wherein said amplifier module (12; 112) and receiver module (14; 114) are detachably attached to each other along the longitudinal extension of the device body.

2. The hearing device (10) according to claim 1, wherein the receiver (18) is incorporated into the receiver module (14), wherein the acoustical output (20) of the electro-acoustical converter is connected to the ear canal plug (16) via a sound tubing (22).

3. The hearing device (100) according to claim 1, wherein the receiver (118) is incorporated into the ear canal plug (116), wherein the acoustical output (120) of the electro-acoustical converter is exposed to the ear canal, and the electrical input thereof is connected to the receiver module (114) by means of electrical wires (122).

4. The hearing device (10; 100) according to claim 1, wherein the amplifier module (12; 112) and the receiver module (14; 114) are attached to each other such that, if the device body is worn by the user behind the ear, the amplifier module (12; 112) is arranged substantially oriented upwards.

5. The hearing device (10; 100) according to claim 1, wherein the amplifier module (12; 112) comprises at least one of an amplifier (26; 126), a battery (30; 130), a microphone system (28′, 28″; 128′, 128″) and user controls.

6. The hearing device (10; 100) according to claim 5, wherein the microphone system (28′, 28″; 128′, 128″) comprises at least one microphone disposed substantially on the top of the amplifier module (12; 112).

7. The hearing device (10) according to claim 1, wherein the receiver module (14; 114) has a customized shape.

8. The hearing device (10) according to claim 2, further comprising at least one sub-microphone mounted to the sound tubing (22) such to receive sound from inside the ear canal and/or from the vicinity of the concha.

9. The hearing device (10; 100) according to claim 1, wherein the receiver module (14; 114) further comprises retention means adapted to detachably attach the receiver module (14; 114) to the amplifier module (12; 112).

10. The hearing device (10; 100) according to claim 1, wherein the receiver module (14; 114) further comprises electrical connection means (24; 124) interfacing the receiver module (14; 114) with the amplifier module (12; 112).

11. The hearing device (10; 100) according to claim 1, further comprising a functionality module (32) detachably attached to at least one of the receiver module (14; 114) and the amplifier module (12; 112), wherein the functionality module (32) includes a rechargeable system.
